(19) United States
(12) Patent Application Publication
(10) Pub. No.: US 2016/0002144 A1
(43) Pub. Date: Jan. 7, 2016

(54) HIGH REFRACTIVE INDEX POLYMER COMPOSITION FOR OPTHALMIC APPLICATIONS

(71) Applicant: Contamac Limited, Saffron Walden (GB)

(72) Inventors: Timothy Charles Higgs, Cambridge (GB); Richard Alexander Young, Linton (GB)

(21) Appl. No.: 14/854,836

(22) Filed: Sep. 15, 2015

Related U.S. Application Data
(62) Division of application No. 13/582,559, filed on Sep. 4, 2012, now abandoned.

(30) Foreign Application Priority Data
Mar. 1, 2010 (GB) GB1003404.9

(51) Int. Cl. C07C 69/54; B29C 39/00; C08F 220/68

(52) U.S. Cl. CPC C07C 69/54; C08F 220/68; B29C 39/006; B29K 2033/04

(57) ABSTRACT

A monomer for a polymerisable composition is described, the monomer having the formula (I):

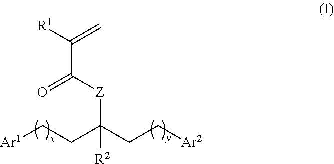

wherein:
—R¹ is —H or alkyl;
—Z— is —O—, —NH— or —NR—, where —R is optionally substituted alkyl or C₅₋₁₀ aryl;
—Ar¹ and —Ar² are each independently optionally substituted C₅₋₁₀ aryl;
—R² is —H, or optionally substituted alkyl or C₅₋₁₀ aryl; and
x and y are each independently 1 to 4. Also described are polymers formed from the composition, and ophthalmic lens products.

### CROSS-REFERENCE TO RELATED PATENT APPLICATIONS

This application is a Divisional application of and claims priority from copending U.S. application Ser. No. 13/582,559, filed Sep. 4, 2012 and titled High Refractive Index Polymer Composition For Opthalmic Applications, which claims priority from expired PCT Application No. PCT/GB 11/00270, filed Feb. 28, 2011 and titled High Refractive Index Polymer Composition For Opthalmic Applications, which claims priority from foreign Application No. GB 1003404.9 filed on Mar. 1, 2010 and titled High Refractive Index Polymer Composition For Opthalmic Applications. The above patent applications are hereby incorporated by reference in their entirety.

### TECHNICAL FIELD

This invention pertains generally to polymerisable monomers and compositions for use in the preparation of polymer compounds. The polymers are suitable for use as ophthalmic lenses.

### BACKGROUND

Contact and intraocular ophthalmic lenses are devices for correcting defective vision. In particular, it has become commonplace to replace cataractous lenses with intraocular lenses (IOLs) using surgical procedures.

A typical surgical procedure for lens replacement involves disintegrating the patient's cataractous natural lens by ultrasonication, aspirating the fragmented lens pieces from the patient's eye through a corneal incision, and then inserting an IOL into the eye through the same incision. In order to reduce surgical trauma, it is advantageous to minimise the size of the incision. For this reason, foldable IOLs have been developed which can be shaped into a small package for insertion through the incision and which unfold into a final shape after being located in the eye.

A significant class of foldable IOLs are formed from flexible polymers which are capable of slowly unfolding at the temperature of the eye (ie. about 37° C.) into an appropriate lens shape.

Hydrophobic acrylic-based polymers have been used for forming flexible IOLs of this type, eg., as disclosed by U.S. Pat. No. 5,674,960, U.S. Pat. No. 5,922,821 and WO 96/40303. Such polymers are deformable, and have relatively high refractive indices (which enables the fabrication of thinner IOLs without concomitantly sacrificing optical refractory power). The overall dioptric power of the IOL depends on both the shape of the optic portion of the lens and the refractive index of the material from which the lens has been made.

There are two conventional manufacturing protocols for hydrophobic-acrylic based IOLs. The first, which is suited to high volume output, involves a one-step moulding process whereby the lens shape is pre-determined by the shape of the mould holding the monomeric components used to fabricate the polymer. The second process involves fashioning a cylindrical ‘blank’ of the lens polymer into the required form using a high resolution lathing system combined with the milling of the haptics for a one-piece IOL design or alternatively the fixing of separately fabricated haptics for a three-piece IOL. Hydrophobic-acrylic polymers capable of yielding IOLs that are easily foldable at room temperature exhibit relatively low glass transition temperatures, T_{g}, typically of less than 20° C. This necessitates the use of specialist lens fabrication equipment, such as cryo-lathes and cryo-mills, to cool the material to below its T_{g} during machining. The tooling addresses a firm non-pliable surface which ensures a high resolution optical quality output.

The glass transition temperatures, T_{g}, for the foldable hydrophobic-acrylic based polymers are generally slightly lower than room temperature (ca. 20° C.) so that they are easily deformable at this temperature without causing physical damage to the polymer, for example by inducing creep, stress or fissures. On immersion in an aqueous environment for a prolonged period of time (eg. vitreous humour in the posterior cavity of the eye) such low T_{g} hydrophobic polymers can be prone to the development of small “glistening formations” in the body of the polymer. This occurs where temperature variations in a hydrophobic polymer can induce spinodal decomposition wherein small amounts of water entrained within the polymer matrix can “condense” on cooling, forming small water-filled cavities. These so-called vacuoles have a considerably lower refractive index (n_{D}²⁰=1.333) than the surrounding hydrophobic polymer matrix (typical n_{D}²⁰≧1.48) and therefore act as light scattering loci and appear to “glisten”.

A number of strategies have been employed to try to prevent the development of glistening bodies in hydrophobic-acrylic based polymers. The presence of glistening bodies is conventionally considered undesirable for this class of IOLs. Strategies to minimise vacuole formation have mostly concentrated on the modulation of the hydrophobicity of the polymer matrix through the inclusion of hydrophilic components (monomers, cross-linkers) into the root formulation. Through this approach it is anticipated that the polymer matrix can more effectively accommodate water, thereby reducing the propensity for glistening body formation.

Pushing this approach to its limits would ultimately yield a hydrogel polymer with appreciable water content. To clarify the distinction between a hydrogel and a polymer matrix accommodating water, US 2001/0003162 states that acrylic materials that absorb 5 wt % or less water at 37° C. are considered to be non-hydrogel acrylic materials.

For example; U.S. Pat. No. 6,852,793 introduces N,N′-dimethylacylamide, and U.S. Pat. No. 7,789,509, U.S. Pat. No. 5,693,095 and WO 2006/063994 each introduce 2-hydroxyethyl methacrylate into predominantly hydrophobic acrylic polymer formulations to enhance the water compatibility of the polymer matrix. In this way glistening body formation may be inhibited, although the maximum 5 wt % equilibrium water content (the “hydrogel threshold”) may be surpassed.

An additional advantage of increased water content is the plasticizing effect of the imbibed water, which conveniently enables the dehydrated polymer to be harder during difficult mechanical processing steps (such as lathing or milling). U.S. Pat. No. 7,790,825 employs a slightly different approach to enhance the water compatibility of a hydrophobic-acrylic polymer. A ‘matrix hydrophilic modulation’ approach is undertaken by addition into the matrix of non-polymerisable block co-polymer surfactants, such as the Pluronic (BASF) range of poloxamers.

Some of the physical properties of the polymer used to make the IOL are dependent on the chemical structure of the monomer. For hydrophobic polymers based on acrylate or methacrylate monomers, the chemical functional group attached to the oxygen atom of the acryl- or methacryl-ester unit can influence the polymer's physical characteristics. In particular, a chemical functional group, which is known to impart particular physical characteristics to the resulting polymer, is covalently attached to the ester unit of the monomer by a bridging group, such as an alkyl chain. For example, U.S. Pat. No. 5,290,892, U.S. Pat. No. 5,403,901, U.S. Pat. No. 5,674,960 and U.S. Pat. No. 5,861,031 all disclose the attachment of an aromatic ring to the terminus of the alkyl bridging chain in order to impart a higher refractive index onto the monomer and the polymer formed from it. Furthermore these patents also disclose the insertion of heteroatoms such as sulfur, nitrogen or oxygen between the bridging alkyl-chain and the aromatic ring which for sulfur imparts additional hydrophobicity and higher refractive index onto the resultant monomer. This heteroatom concept is further developed in WO 00/79312 which discloses several classes of acrylate or methacrylate based monomers that can be used to form homopolymer or copolymer compositions for the manufacture of IOL implants. The disclosed monomers contain an aryl functional group attached to the ester by an alkyl chain bridge where the alkyl bridging group may optionally also contain one or more oxygen or sulfur heteroatoms. Where the alkyl-chain bridge comprises multiple heteroatoms, these heteroatoms are dispersed evenly along the alkyl-chain in a polyether or polythioether motif. Where the alkyl-chain bridge comprises a single heteroatom, this atom forms an interlink link between the aryl and alkyl groups. For example, where the heteroatom is an oxygen atom, the group is an arylalkylether motif. Copolymers containing phenylthioethyl acrylate (ie. an acrylate with an arylthio-alkyl side chain) were prepared and characterised in WO 00/79312.

EP 1,792,923 and WO 2007/094665 disclose acrylic monomers possessing heteroatom arylalkylether or arylalkylthioether motifs where the refractive index is further amplified through the incorporation of more than one (typically two) arylalkylether or arylalkylthioether arms onto the core acryl- or methacryl-ester polymerisable functionality. The synthesis of the dual arm arylalkylthioether monomer, 1,3-bis(phenylthio)propan-2-yl methacrylate is disclosed in EP 1,792,923 and WO 2007/094665, as well as its use in preparing high refractive index hydrophobic polymer compositions.

The present invention is based on the finding that hydrophobic acrylic-based polymers having improved properties can be obtained from a class of acrylate, alkylacrylate, acrylamide or alkylacrylamide based monomers that have substituents located at particular positions on plural-arm bridging groups.

### SUMMARY OF THE INVENTION

The present invention provides monomers and polymerisable compositions for use in the preparation of polymers for use in ophthalmic lenses and blanks for the same. The monomers of the invention may be used to prepare polymers having improved optical characteristics, such as greater refractive index, and/or improved physical characteristics, such as lower glass temperature (T_{g}). Such polymers are suitable for use in ophthalmic lenses.

A first aspect of the present invention provides a monomer for a polymerisable composition, the monomer having the formula (I):

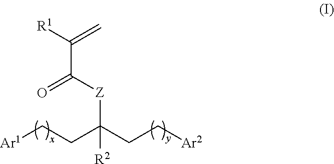

wherein:
—R¹ is —H or alkyl;
—Z— is —O—, —NH— or —NR—, where —R is optionally substituted alkyl or C₅₋₁₀ aryl;
—Ar¹ and —Ar² are each independently optionally substituted C₅₋₁₀ aryl;
—R² is —H, or optionally substituted alkyl or C₅₋₁₀ aryl; and
x and y are each independently 1 to 4.

In one embodiment, each C₅₋₁₀ aryl is C₅₋₆ aryl.

In one embodiment, each C₅₋₁₀ aryl or C₅₋₆ aryl absorbs a negligible amount of electromagnetic radiation having a wavelength in the range 300-900 nm.

In one embodiment, each C₅₋₁₀ aryl is a C₆₋₁₀ carboaryl.

Where —R¹ is alkyl and —Z— is —O—, the monomer may be referred to as an alkylacrylate monomer. Where —R¹ is —H and X is —O—, the monomer may be referred to as an acrylate monomer.

Where —R¹ is alkyl and —Z— is —NH— or —NHR—, the monomer may be referred to as an alkylacrylamide monomer. Where —R¹ is —H and X is —NH— or —NHR—, the monomer may be referred to as an acrylamide monomer.

In a second aspect of the invention there is provided a polymerisable composition comprising one or more monomers of formula (I).

In a third aspect of the invention there is provided a polymer obtained or obtainable from a polymerisable composition comprising a monomer of formula (I). In one embodiment, the polymer is a polymer formed from the polymerisable composition comprising a monomer of formula (I).

In a fourth aspect of the invention there is a provided a method for the synthesis of a polymer, the method comprising the step of polymerising a polymerisable composition comprising a monomer of formula (I).

In a fifth aspect of the invention there is provided a blank for an ophthalmic lens formed from the polymer of the third aspect of the invention.

In a sixth aspect of the invention there is provided an ophthalmic lens formed from the polymer of the third aspect of the invention.

Other aspects of the invention provide methods for the preparation of the blank of the fifth aspect of the invention and the ophthalmic lens of the sixth aspect of the invention.

The invention also provides the use of the polymer of the third aspect of the invention as an intraocular lens.

### BRIEF DESCRIPTION OF THE DRAWING

FIG. 1 is an electronic absorption spectrum showing the percentage transmittance of light for three samples at wavelengths in the range 200 to 500 nm. The solid spectral line is for a sample of neat ethylbenzene, the dotted spectral line is for a sample of thioanisole, and the dash-and-dot spectral line is the transmittance curve of the human cornea. The regions of the ultraviolet spectrum UVA (approx. 320 to 400 nm), UVB (approx. 290 to 320 nm) and UVC (approx. 100 to 290 nm) are shown as the horizontally lined, clear and the diagonally lined regions respectively.

### DETAILED DESCRIPTION OF THE INVENTION

The present invention provides a monomer of formula (I) for use in a polymerisable composition. The monomer is provided with at least two aryl groups, —Ar¹ and —Ar², attached at the termini of respective alkyl spacers. Changes to one, or both, of the aryl groups may modulate the absorption properties of a polymer fabricated from the monomer. Each aryl group is connected to a fulcrum carbon atom via alkyl spacer groups that are uninterrupted by heteroatom functionality. The fulcrum carbon atom is itself directly attached to the group —Z—, which is part of the polymerisable portion of the monomer compound. The monomer may be regarded as having two arms, each connecting the aryl groups to the polymerisable region of the monomer via the fulcrum carbon atom.

In contrast, the prior art describes compounds having alkyl spacer groups that are interrupted with heteroatom functionality, such as sulfur or nitrogen atoms. Examples include monomers having single arms, such as described in WO 00/79312, U.S. Pat. No. 5,290,892, U.S. Pat. No. 5,403,901, U.S. Pat. No. 5,674,960, U.S. Pat. No. 5,861,031, and monomers having multiple arms, such as described in EP 1,792,923 and WO 2007/094665.

Many of the monomer compounds described in art have a single alkyl arm linking the aryl functionality with the polymerisable region of the monomer, including, for example, U.S. Pat. No. 5,693,095, U.S. Pat. No. 6,780,899, U.S. Pat. No. 6,241,766, U.S. Pat. No. 6,271,281, U.S. Pat. No. 6,281,319, U.S. Pat. No. 6,326,448, and WO 00/79312. The present invention provides a monomer having two arms interlinking at least two aryl functionalities as described above, and the inventors have established that such monomers may be used to prepare polymers having a greater refractive index than is possible in those monomer compounds having a single alkyl arm linked to a single aryl functionality.

Certain monomer compounds described in the prior art comprise aryl groups that are connected to an alkyl spacer group through electron-donating heteroatom functionality, such as sulfur and nitrogen. For such monomers the absorption characteristics of the chromophore are altered such that significant amounts of UVB (290-320 nm) and even UVA (320-400 nm) radiation are absorbed. In the absence of the heteroatom functionality, a single-ring aryl group would be expected to absorb predominantly UVC (100-290 nm) radiation.

The absorption of UVB and UVA radiation may compromise the long term stability of a polymer containing this functionality through photooxidative degradation phenomena. Conversely, solar UVC is not considered to pose a significant obstacle to achieving long-term polymer stability as it is almost entirely absorbed by stratospheric ozone.

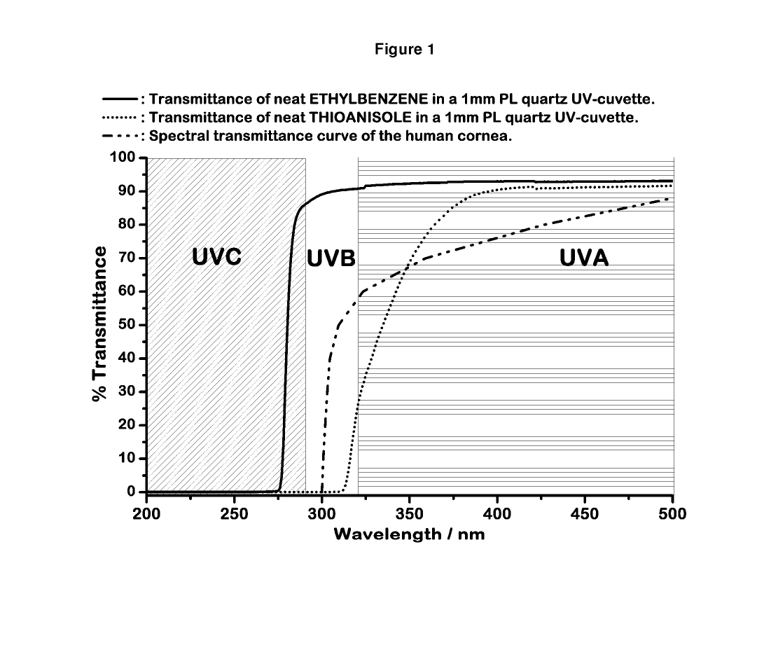

The influence of heteroatom functionality on the absorption/transmittance properties of an aryl group is shown in FIG. 1. In this FIGURE, the spectral transmittance curve for neat ethylbenzene is shown, representing the chromophore of an “Ar” group, in this instance phenyl, in a monomer of the invention absent the fulcrum carbon atom and absent the polymerisable component of the monomer (for example, absent the acrylate or alkylacrylate component). Also shown in FIG. 1 is the spectral transmittance curve for thioanisole, representing the chromophore of an “Ar” group in a monomer absent a polymerisable component (for example, absent an acrylate or an alkylacrylate component) where an aryl group is connected to an alkyl arm via sulfur heteroatom functionality. The transmittance curves were obtained from neat samples contained within a 1 mm path-length quartz cuvette analysed at wavelengths over the range 200-500 nm. Overlaid onto the spectral curves is the transmission curve of a human cornea over the corresponding wavelengths.

FIG. 1 shows that the thioanisole compound absorbs a significant amount of UVA and UVB radiation (≧300 nm) that would otherwise be transmitted through the human cornea. Prolonged exposure of a polymer derived from a monomer having thioanisole functionality would likely result in radiation damage to that polymer.

In contrast, FIG. 1 shows that an ethylbenzene chromophore absorbs a negligible amount of UVA and UVB radiation, and hence effectively no light at wavelengths ≧300 nm. Consequently, a polymer derived from a monomer containing one or more ethylbenzene-derived units would exhibit considerable resistance to radiative damage when subjected to corneal-filtered UV/Visible light, for example in a pseudophakic posterior chamber intraocular lens.

The group —R² may be selected so as to provide a polymer having certain physical and optical properties. For example, where —R² is optionally substituted aryl, or —R² comprises an aryl group (for example where —R² is alkyl substituted with aryl), the refractive index of the resulting polymer may be amplified. Conversely, where —R² is an optionally substituted long chain alkyl group, for example where —R² is C₈₋₂₀ alkyl, the refractive index of the resulting polymer may be down-modulated.

Additionally, where —R² is a rigid group, or —R² comprises a rigid group, for example where —R² is or comprises an aryl group, the T_{g} of the resulting polymer may be increased. Conversely, where —R² is or comprises a flexible chain, for example where —R² is or comprises an alkyl group, the T_{g} of the resulting polymer may be decreased.

The refractive index of a polymer may be increased without concomitant increase in T_{g} by employing a monomer where —R² comprises both alkyl and aryl functionalities, for example where —R² is C₁₋₆ alkyl substituted with aryl. It is believed that the alkyl group offsets the increase in T_{g} imparted by the incorporation of a rigid aromatic ring into the monomer. The length of the alkyl group may modulate the overall T_{g} of the polymer with a longer chain lowering the polymer T_{g} and a shorter chain conferring a lesser offsetting effect, and thereby resulting in a higher T_{g} for the resulting polymer. It is preferred that —R² is —H.

The value of x and/or y may be selected so as to provide a polymer product having a particular T_{g}. Generally, where the length of either or both of the arms is increased, i.e. where the value of x and/or y is increased, the value of T_{g} for the resulting polymer will be decreased. Thus changes in x and/or y permits modulation of the overall flexibility (foldability) of the polymer product. Preferably, the value of x and y is in the range 1 to 4, in the range 1 to 3, or both are 1. Most preferably both x and y are 1.

In some embodiments, the compound may have a chiral centre. For example, when the values of x and y are different, and/or —Ar¹ and —Ar² are different, the resulting monomer encompassed by formula (I) may be optically active. The chiral centre, or each chiral centre, if more than one is present, is independently in the R-configuration or the S-configuration. If no configuration is indicated, then both configurations are encompassed.

### Polymerisable Composition

In a second aspect of the invention there is provided a polymerisable composition comprising one, or more, of the monomers defined in the first aspect of the invention. The present inventors have established that polymer ophthalmic lenses, particularly IOLs, formed from such a composition may be suitably flexible to be folded or rolled to a size suitable for surgical insertion.

The polymerisable composition of the invention has a first monomer comprising one or more monomers of formula (I). The polymerisable composition may have from 5 to 99 wt % of the first monomer. The remaining portion of the polymerisable composition may comprises other monomer components and/or conventional polymerisation agents as described below.

Preferably the bottom of the range is 20, 30, 40 or 50 wt %.

Preferably the top of the range is 95, 98 or 99 wt %.

For example, in one embodiment, the range is from 50 to 99 wt %.

The polymerisable composition of the invention may further comprise one or more of a second monomer, a third monomer and a fourth monomer for copolymerisation with the first monomer. The second, third and/or fourth may be used to adjust the physical and/or optical properties of the polymer formed from the composition, as described below.

The polymerisable composition may have from 0 to 50 wt % of the second monomer.

Preferably the bottom of the range is 1, 3, 5 or 10 wt %.

Preferably the top of the range is 30, or 40 wt %.

The polymerisable composition may have at least 5 wt % of the second monomer.

In one embodiment, the second monomer is a monomer having an acrylate or methacrylate group.

Examples of second monomers include, but are not limited to, methyl acrylate, ethyl acrylate, propyl acrylate, butyl acrylate, hexyl acrylate, cyclohexyl acrylate, ethoxyethyl acrylate, methoxyethyl acrylate, methyl methacrylate, ethyl methacrylate, propyl methacrylate, butyl methacrylate, hexyl methacrylate, cyclohexyl methacrylate, ethoxyethyl methacrylate, methoxyethyl methacrylate, isobornyl methacrylate, isobornyl acrylate, phenylethyl methacrylate and phenylethyl acrylate and mixtures thereof.

The third monomer is a hydrophilic monomer. The third monomer is suitable for polymerisation with the first monomer and/or the second monomer, where present.

The polymerisable composition may have from 0 to 50 wt % of the third monomer.

Preferably the top of the range is 15, 25, or 40 wt %.

For example, in one embodiment, the range is from 0 to 15 wt %.

The polymerisable composition may have at least 0.1, 0.5 or 1 wt % of the third monomer.

The third hydrophilic monomer may be incorporated into the polymer to alter, for example to down-modulate, the refractive index of the polymerised article and/or to control the mechanical properties of the polymer product through the plasticising effect of water. The inclusion of a hydrophilic monomer into the root formulation can also be strategically employed to modulate the hydrophilicity of the polymer matrix, thereby reducing the propensity of the material to glistening body formation.

Examples of third monomers include, but are not limited to, 2-hydroxyethyl methacrylate, 2-hydroxyethyl acrylate, 2-hydroxypropyl methacrylate, 2-hydroxypropyl acrylate, 3-hydroxypropyl methacrylate, 3-hydroxypropyl acrylate, 4-hydroxybutyl methacrylate, 4-hydroxybutyl acrylate, N-vinyl pyrrolidin-2-one, methacrylic acid, acrylic acid, acrylamide, methacrylamide, N,N′-dimethyl acrylamide, N-methyl-N-vinylacetamide, 2-hydroxy-3-phenoxypropyl acrylate, glycerol monomethacrylate, polyethylene oxide monomethacrylate (preferably M_{w}=200-400) and N-(2-hydroxypropyl) methacrylamide, and mixtures thereof. The preferred third monomers are 2-hydroxyethyl methacrylate and 2-hydroxyethyl acrylate.

The fourth monomer is a crosslinking monomer. The crosslinking monomer is suitable for forming crosslinks with monomers in the polymerisable composition. Typically, the fourth monomer is provided with two or more reactive functional groups for reaction with suitable functionality on the first monomer, and/or the second monomer, and/or third monomer, where present. The fourth monomer may be provided with functional groups for cross-reactivity between fourth monomer molecules.

The polymerisable composition may have at least 0.1, 0.8, 1.5 or 3 wt % of the fourth monomer.

Preferably the reactive functional groups of the fourth monomer are unsaturated functional groups such as double or triple bonds. The fourth monomer may be used to generate a three dimensional polymeric network in the polymerised product. The level of cross-linking monomer in the polymerisable composition may be adjusted to alter the material properties of the resulting polymer, most particularly the flexibility and elongation to break parameters.

Examples of fourth monomers include, but are not limited to, ethylene glycol dimethylacrylate, ethyleneglycol diacrylate, diethylene glycol dimethylacrylate, diethylene glycol diacrylate, allyl acrylate, allyl methacrylate, 1,3-propanediol dimethacrylate, di-allyl maleate, 1,4-butanediol dimethacrylate and 1,4-butanediol diacrylate, 1,3-propanediol diacrylate, 1,3-propanediol dimethacrylate, 1,4-butanediol dimethacrylate, 1,6-hexanediol diacrylate, 1,6-hexanediol dimethacrylate, triethylene glycol diacrylate, triethylene glycol dimethacrylate, neopentyl glycol diacrylate, neopentyl glycol dimethacrylate, butylene glycol dimethacrylate, butylene glycol diacrylate, thio-diethylene glycol diacrylate, thio-diethylene glycol dimethacrylate, trimethylolpropane triacrylate, and diacrylates and dimethacrylates of bisphenol A, bisphenol A ethoxylate (1-3EO/phenol), bisphenol A propoxylate (1-3EO/phenol). Other crosslinking fourth monomers include N,N′-dihydroxyethylene bisacrylamide, diallyl phthalate, triallyl cyanurate, divinylbenzene, ethylene glycol divinyl ether, N,N-methylene-bis-(meth)acrylamide, sulfonated divinylbenzene, divinylsulfone and 1,3,5-triallyl-1,3,5-triazine-2,4,6(1H,3H,5H)-trione.

The composition may further comprise conventional compounds including, but not limited to, a thermally- or light-activated polymerisation initiator (preferably in an amount of up to 5% by weight of the composition), a “fixable”, for example by free-radical vinyl-polymerisation, UV-light absorber (preferably in an amount of up to 5% by weight of the composition), a “fixable” blue-light absorber (preferably in an amount of up to 0.5% by weight of the composition), a tackiness modifying agent, a strengthening agent, or a combination thereof. In one embodiment, the conventional compound comprises a functional group that is suitable for polymerisation with the first monomer and/or the second, third and fourth monomer where present.

As used herein, the term “fixable” is used in relation to a compound that may be incorporated into the polymer upon polymerisation of the polymerisable composition. Thus, a fixable compound is suitable for reaction with one or more of the first, second, third and fourth monomers, where present. Exemplary fixable monomers include those having vinyl functionalities for participation in, for instance, free-radical polymerisation with other vinyl-containing monomers, such as the first monomer described herein.

Examples of suitable UV-light absorbers include, but is not limited to, compounds comprising the benzoylphen-2-ol or 2-(2H-benzo[d][1,2,3]triazol-2-yl)phenolchromophore, such as 2-[3′-(2′ H-benzotriazol-2′-yl)-4′-hydroxyphenyl]-ethylmethacrylate, 2-(4′-benzoyl-3′-hydroxyphenoxy)ethyl acrylate, 2-hydroxy-4-allyloxybenzophenone, 2-(2′-hydroxy-5-methacryloxyethylphenyl)-2H-benzotriazole, 13-(4-benzotriazoyl-3-hydroxyphenoxy)ethylacrylate, 4-(2-acryloxyethoxy)-2-hydroxybenzophenone, 4-methacryloyloxy-2-hydroxybenzophenone, 2-(2′-methacryloyloxy-5′-methylphenyl)benzotriazole, 2-(2′-hydroxy-5′-methacryloxyethylphenyl)-2H-benzotriazole, 2-[3′-tert-butyl-2′-hydroxy-5′-(3″-methacryloyloxypropyl)phenyl]-5-chlorobenzotriazole, 2-(3′-tert-butyl-5′-(3″-dimethylvinylsilylpropoxy)-2′-hydroxyphenyl]-5-methoxybenzotriazole, 2-(3′-allyl-2′-hydroxy-5′-methylphenyl)benzotriazole, 2-[3′-tert-butyl-2′-hydroxy-5′-(3″-methacryloyloxypropoxy)phenyl]-5-methoxybenzotriazole, 2-[3′-tert-butyl-2′-hydroxy-5′-(3″-methacyloyloxypropoxy)phenyl]-5-chlorobenzotriazole, 2-(2′-hydroxy-5′-methacryloyloxyethylphenyl)-2H-benzotriazole and 2-(2′-hydroxy-3′-methallyl-5′-methylphenyl)benzotriazole. A preferred monomer as a UV-light absorber is 2-[3′-(2′H-benzotriazol-2′-yl)-4′-hydroxyphenyl]-ethylmethacrylate.

UV-blocker molecules, such as those described herein, are known in the art to be exceptionally stable to both UVA and UVB solar radiation. The molecules are capable of absorbing light at wavelengths in these spectral ranges, and then dissipating this energy as heat. This dissipation occurs without the induction of potentially deleterious chemical reactivity, such as photooxidation, that could damage the integrity of the polymer.

The incorporation of UV-blocking monomers into a polymerisable composition can greatly extend the lifetime of a polymer subjected to solar radiation. However, the UV-blocking element of a polymer can only partially mitigate the effect of solar radiation if other components within that polymer can “compete” with respect to the absorption of UVA and especially UVB radiation. For example, a monomer entity comprising a thioanisole chromotype, which absorbs light at wavelengths in the UVB region, could participate in destruction phenomena such as photooxidation which are capable of compromising the overall integrity of the polymer.

A strengthening agent is an agent capable of increasing the tensile strength of the resulting polymer, for example by permitting the polymer to elongate a long way before breaking, or requiring a large load on the polymer (not necessarily contingent on having a high elongation before breaking) before it snaps.

One or more tackiness modifying agents may be added to the polymerisable composition according to the present invention. The inclusion of a tackiness modifying component can advantageously yield a more tractable polymer product. A tackiness modifying agent may comprise at least one reactive unsaturated functional group, such as vinyl, acrylate or methacrylate-based groups.

Examples of tackiness modifying agents include, but is not limited to, fluorocarbon acrylates and methacrylates such as hexafluoro-iso-propyl methacrylate, 1H,1H,7H-dodecafluoroheptyl methacrylate, 1H,1H-heptafluorobutyl acrylate, 1H,1H,3H-hexafluorobutyl methacrylate, 1H,1H,5H-octafluoropentyl methacrylate, 2,2,2-trifluoroethyl acrylate, and linear-chain alkyl acrylates or methacrylates such as butyl acrylate, butyl methacrylate, pentyl acrylate, pentyl methacrylate, hexyl acrylate, hexyl methacrylate, heptyl acrylate, heptyl methacrylate, octyl acrylate, octyl methacrylate and/or branched-chain alkyl acrylates or methacrylates such as isopentyl acrylate, isopentyl methacrylate, isobutyl acrylate, isobutyl methacrylate, 2,2-dimethylpropyl acrylate, 2,2-dimethyl propyl methacrylate, 2-ethylhexyl acrylate and 2-ethylhexyl methacrylate.

The polymerisable composition may comprise a thermally- or light-activated polymerisation initiator. Preferably, the initiator is a free-radical polymerisation initiator.

In a preferred embodiment, the polymerisable composition comprises 0.01 to 0.50 wt % of the polymerisation initiator.

Free-radical polymerisation may be initiated thermally by using a thermal free radical initiator such as peroxide, peroxidedicarbonate or azo-based initiators. Examples of peroxide or peroxidedicarbonate based initiators include, but are not limited to, dilauroyl peroxide, didecanoyl peroxide, tert-butyl peroxyneodecanoate, di(4-tert-butylcyclohexyl) peroxydicarbonate, dicetyl peroxydicarbonate, dimyristyl peroxydicarbonate. Examples of azo-based initiators include, but are not limited to, 1,1′-azobiscyanocyclohexane, 2,2′-azobis(2,4-dimethylvaleronitrile), 2,2′-azobisisobutyronitrile and 2,2′-azobis(2-methylbutyronitrile).

Photointiated free-radical polymerisation may be carried out in the presence of a photoinitiator, such as CIBA's Irgacure® 1800 [comprising 25% bis(2,6-dimethoxybenzoyl)-2,4,4-trimethyl-pentylphosphineoxide and 75% 1-hydroxy-cyclohexyl-phenyl-ketone], Irgacure® 184 [comprising 100% 1-hydroxy-cyclohexyl-phenyl-ketone], Irgacure® 819 [comprising 100% bis(2,4,6-trimethylbenzoyl)-phenylphosphineoxide], Irgacure® 2959 [comprising 100% 1-[4-(2-hydroxyethoxy)-phenyl]-2-hydroxy-2-methyl-1-propane-1-one], Darocur® MBF [comprising 100% phenyl glyoxylic acid methyl ester], Darocur® TPO [comprising 100% 2,4,6-trimethylbenzoyl-diphenyl-phosphineoxide] and Darocur® 1173 [comprising 100% 2-hydroxy-2-methyl-1-phenyl-propan-1-one].

In instances where thermal polymerization is employed to fabricate the hydrophobic-acrylic polymer composition, the preferred free-radical initiator is 2,2′-azobisisobutyronitrile (AIBN). Where photo-initiated free-radical polymerization is employed to fabricate the hydrophobic-acrylic polymer composition, the preferred initiator bis(2,4,6-trimethylbenzoyl)-phenyl-phosphineoxide (for example, Irgacure 819).

The polymerisable composition may further comprise a diluent. The diluent may aid the processing of the polymer after polymerisation, particularly during the expulsion of extractable contaminants, such as residual monomers, by treatment with an appropriate solvent. A pre-swelled polymer network of the polymer having an incorporated diluent facilitates the removal of residual, leachable contaminants from the body of the polymer. Solvent extraction of a dry polymer typically causes swelling of the polymer body which can lead to a degradation of mechanical properties, this effect can be mitigated through the “pre-swelling” of the polymer network with a diluent at an appropriate level.

The polymerisable composition may comprise from 2 to 40 wt % of the diluent.

Preferably the top of the range is 25, 30 or 35 wt %.

Preferably the bottom of the range is 2, 5 or 10 wt %.

For example, in one embodiment, the range is from 2 to 30 wt %.

Examples of suitable diluents include, but are not limited to, ethylene glycol, di(ethylene glycol), tetra(ethylene glycol), glycerol, 1,5-pentanediol, ethylene glycol monomethyl ether, ethylene glycol dimethyl ether, triethylene glycol monomethyl ether, 2-ethoxyethanol, solketal, benzonitrile, hexamethylphosphoramide, N-methylpyrrolidin-2-one and N,N′-dimethylformamide. Preferred diluents for inclusion in the polymerisable compositions of the present invention are N-methylpyrrolidin-2-one and N,N′-dimethylformamide.

The total amount of first monomer, second, third and fourth monomer, where present, conventional compounds, where present, and diluent, where present, does not exceed 100 wt %.

Polymers and Methods for their Preparation

In a third aspect of the invention there is provided a polymer obtained or obtainable from a polymerisable composition comprising a monomer of formula (I). The polymers are suitable for use in implantable medical devices, including ophthalmic devices such as IOLs.

The polymer of the invention is a polymer obtainable by polymerisation of a polymerisable composition of the invention. In one embodiment, the polymer is obtained or obtainable by free radical polymerisation of a polymerisable composition of the invention.

The polymers of the invention may comprise one or more units of formula (II):

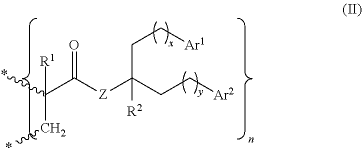

where —R¹, —Z—, —Ar¹, —Ar², —R², and x and y are as defined for the monomers of formula (I).

In one embodiment, the polymer contains one or more units of formula (II).

In one embodiment, the amount of unit (II) present in the polymer as a mole fraction of all the units present, is at least 0.40. In one embodiment, the mole fraction is at least 0.60, at least 0.80, at least 0.90, or at least 0.95. The final mole fraction of (II) in the polymer may be altered by, for example, increasing or decreasing the amount of monomer of formula (I) in the polymerizable composition.

In one embodiment, the number average of units of (I) present in the polymer is at least 100, or is at least 500, or is at least 1,000, or is at least 5,000.

In one embodiment, the average molecular weight of the polymer is at least 25,000 Da, or is at least 125,000 Da, or is least 250,000 Da, or is at least 1,250,000 Da.

In one embodiment, the polymer has a T_{g} in the range of from −50 to 35° C., preferably −20 to 30° C., or more preferably −15 to 25° C.

T_{g} may be measured by dynamic mechanical thermal analysis (DMTA) as is well known to those skilled in the art.

In one embodiment, the polymer has an elongation at 20° C. of at least 50%, and preferably of at least 75%.

In one embodiment, the polymer has an elongation at 20° C. of from 50% to 250%, and preferably from 125% to 200%.

The elongation to break may be measured by tensile testing of a sample using a tensiometer, as is known to those skilled in the art.

In one embodiment, the polymer has a T_{g} of less than 25° C. and an elongation to break of at least 140%.

In one embodiment, the polymer has a refractive index at 20° C. of at least 1.50. It is preferred that the polymer has a refractive index of at least 1.50 and has an equilibrium water content in the range of 0 to 50 wt %.

The refractive index may be measured with an Abbe refractometer as is known to those skilled in the art.

Products and Methods for their Manufacture

The ophthalmic lens of the invention is preferably an intraocular lens (IOL). Such lenses can either be described as phakic, aphakic or pseudophakic. A phakic lens is implantable in the eye without removal of the natural crystalline lens in a procedure to improve vision in patients with larger visual errors than typically seen in the general population. However an aphakic lens is implanted after removal of a clear crystalline lens with the goal again being an improvement in near or distance vision. Both these cases are examples of refractive surgery. A pseudophakic lens, the most common type of IOL, is used when the natural crystalline lens has been removed after developing a cataract. This procedure is the basis of cataract surgery. In addition to the types of lenses described, the placement of the lens within the eye is also used to describe the type of IOL implanted in patients. Such lenses are either implanted in the posterior segment of the eye, or the anterior segment of the eye.

Intraocular lenses may comprise optic and haptic portions. The optic portion comprises a mass of refracting material contained between two essentially spherical surfaces and is responsible for determining the visual functionality of the lens. The form of the optic portion (i.e. the curvature of its anterior and posterior surfaces), together with the refractive index of the polymer, determines the dioptric power of the lens. The haptic portion holds the lens in position beneath, and parallel to, the cornea after implantation and a key function of the haptic is to ensure the optic portion remains centred over the central visual zone of the eye. A single piece intraocular lens is manufactured from a single polymer blank and both the optic and haptic portions of the lens are usually formed simultaneously. A two or three piece IOL on the other hand usually comprises an optic portion manufactured from an individual polymer piece and the haptic portion or portions, which are produced from separate polymeric article(s), are subsequently attached to the optic portion in an additional manufacturing step.

The ophthalmic lens of the present invention may be described as having both an anterior surface and a posterior surface. In the case of an IOL, the posterior surface of the lens faces the back of the eye while the anterior surface is directed toward the front of the eye.

Further aspects of the present invention provide a blank for an ophthalmic lens formed from the polymer of the invention and an ophthalmic lens formed from the polymer of the invention.

The blank may be formed as a substantially cylindrical polymer product, with the cylinder typically having a circular diameter exceeding that of the altitude of the cylinder. The substantially cylindrical product may be formed from a cast moulding process using a suitable depression mould. The cylindrical polymer product may be worked, for example machined using milling and/or lathe cutting processes familiar to those skilled in the art, until a finished ophthalmic lens is obtained. The working process may also be referred to as machining of a shaped polymer product.

Alternatively, a mould may be used to fabricate a completely or substantially finished ophthalmic lens directly. Additional machining, typically involving the polishing of the optic portions of the lens, is usually required for a substantially finished ophthalmic lens to produce a useable lens.

The present invention also encompasses methods for fabricating a blank for an ophthalmic lens, and methods for fabricating an ophthalmic lens from a lens blank or from a polymer of a previous aspect of the invention.

A general method for fabricating an ophthalmic lens of the present invention comprises the steps of:
(a) providing a blank according to the present invention; and
(b) working the blank so as to form an ophthalmic lens.

Lens blanks according to the present invention may be manufactured according to any one of the methods described below. Reference to the shape or design of a mould as used herein refers to the shape or design of the part of the mould where the actual polymerisation of the polymer takes place.

A first method of forming a blank for an ophthalmic lens comprises the steps of:
(a) polymerisation of a composition of the present invention in a substantially rod-shaped mould thereby to form a polymer rod; and
(b) working the polymer rod into a plurality of cylindrical blanks.

A polymerisation reaction on a polymerisable composition of the present invention may be performed in the mould to form the polymer. Alternatively a preformed linear polymer may be placed in the mould and then cured to obtain the desired polymer product. An example of polymerisation in the mould is described below in the button moulding method.

A substantially rod-shaped (eg. cylindrical) mould is typically constructed from polypolypropylene, polyethylene, PTFE or glass. The shape and size of the mould determines the diameter of the polymer rod. The diameter for the polymer rod is chosen for the design of the resulting ophthalmic lens to be formed; a larger diameter rod is required for a single piece ophthalmic lens and a smaller diameter rod is sufficient for a two or three-piece design ophthalmic lens. Typically, the polymer rod formed is worked into a series of homogeneous discs as described above. Generally the discs have parallel faces.

In an alternative method, a blank for an ophthalmic lens may be formed in a method comprising the step of polymerisation of a polymerisable composition according to the invention in a button mould thereby forming a lens blank. A polymerisation reaction on the polymerisable composition of the present invention may be performed in the button mould to form the polymer. An uncured polymer may be placed in the mould and cured, as an alternative to this method.

Typically, button moulds consist of an array of button impressions on a pre-formed polypropylene, polyethylene or PTFE sheet. The dimensions of the individual button moulds are determined by the resulting design of the final lens. Button moulds with a larger diameter button are required for a single piece ophthalmic lens, and a smaller diameter button mould is sufficient for a two or three-piece design ophthalmic lens.

The mould-sheet is covered with a lid-stock, typically comprising polyethylene or polypropylene. The lid-stock covered mould-sheet is filled with the polymer composition of the present invention and the mould is sealed, for example using a heat-sealing bar apparatus. A monomer formulation may be polymerised in the mould using an oven or, more preferentially, in a water bath thermally equilibrated to the required polymerisation temperature.

Once the polymerisation step has been completed, the water bath is allowed to cool and the mould-sheet is removed, cleaned and dried. The lid-stock can then be peeled from the mould and the polymerised discs extruded. It may be advantageous to perform the lid-stock removal and mould extrusion at a reduced temperature to prevent possible damage to the relatively soft polymer disc. This is particularly important when diluents are employed in the polymerisable composition. In such instances the mould can be chilled to a temperature lower than that of the freezing point of the diluent (or the T_{g} of the polymer where a diluent is not employed) for a period of 5 to 60 minutes immediately prior to lid-stock removal and subsequent mould extrusion.

Both of the above moulding methods for providing a lens blank may include an additional step of flushing the polymer rod initially formed after the polymerisation step. The flushing step comprises treating the polymer rods or buttons with an appropriate solvent to remove extractable contaminants. An example of a suitable solvent for extracting contaminants is acetonitrile.

It may also be desirable to include a drying step after the polymerisation step, and after any flushing step. The polymer rod or disc may be dried or annealed at an elevated temperature, either in air, an inert atmosphere of nitrogen or argon or under reduced pressure. The drying step may be carried out at a temperature in the range 30 to 150° C. Preferably, the drying or annealing step is performed under reduced pressure in the range 0.001 to 300 torr. Preferably the pressure is in the range 0.01 to 10 torr, most preferably 0.03 to 0.30 torr.

A lens blank obtained using the above moulding methods may be ground and polished such that the dimensions of the disc or blank lies within a stringent tolerance window with respect to the accuracy of both the diameter of the disc and the altitude between the opposing circular faces and their degree of parallelism.

The present invention also provides a method for preparing an ophthalmic lens, wherein a lens blank is lathe cut and optionally machine milled into a required lens shape. The step of machining a blank or polymer disc to form an ophthalmic lens comprises the following steps:
(a) lathe machining a first surface of an ophthalmic lens from a lens blank,
(b) lathe machining a second surface of an ophthalmic lens from the lens blank.
   In some circumstances it may be preferable to first machine the anterior surface of the IOL followed by the posterior surface. Alternatively, the posterior surface may be machined first.

Before each lathe machining step, the lens blank is adhered or blocked onto a brass-chuck or poly(methylmethacrylate) cylinder. This may be achieved by using a low (melting) temperature blocking wax. Depending on the cutting parameters employed, it may be desirable to cool the disc during lathing, to a temperature below its T_{g} in order to increase its hardness, such as with a cold-air stream, including that provided by a vortex cold-air tube or cryogenic air-stream. Additional benefit may also be gained through the use of a cryogenic lathing system where the actual polymer blank and the cutting tool are held at low temperatures during the cutting process.

After each ophthalmic lens surface has been lathe machined into the polymer, the machined surface may be inspected for defects and optical performance. Haptics may then be milled or fitted, depending on the ophthalmic lens design. Typically, the final ophthalmic lens is then inspected for defects.

For example, a typical method of lathe machining an IOL from a lens blank comprises one or more of the following steps:
(i) blocking a lens blank on a brass-chuck or a poly(methylmethacrylate) cylinder using a low temperature blocking wax;
(ii) applying a cold-air stream onto the rotating disc, such as by using a vortex cold-air tube, and lathe machine the first surface of a lens from a lens blank;
(iii) inspect the machined surface for defects. If no defects are present, then de-block the machined lens blank;
(iv) block the first surface of the lens blank onto the chuck using a low temperature blocking wax;
(v) apply a cold-air stream onto the rotating disc, for example, by using a vortex cold-air tube. Then lathe the second surface of the lens optic from the lens blank;
(vi) inspect the machined surface for defects;
(vii) for a one piece IOL design, mill the haptics. A cold-air stream may optionally be applied. For a multi-piece IOL design, attach the IOL haptics;
(viii) de-block the IOL, for example by dissolving the blocking wax with 80-100 petroleum ether;
(ix) polish the IOL to smooth the lens surfaces and the lens edges;
(x) if required, hydrate the IOL in physiological saline;
(xi) inspect the final IOL for defects.

Alternatively, step (x) may be performed prior to step (ix).

The invention also provides a method of preparing an ophthalmic lens of the invention, such as an IOL, by direct formation of a partial or complete lens using a mould designed specifically for that purpose. The method comprises the step of polymerising a polymerizable composition of the present invention in a mould thereby to form an ophthalmic lens, wherein the mould is shaped so as to provide an ophthalmic lens having anterior and/or posterior portions consistent with conferring the desired optical performance (for example, focussing power) onto the polymer article.

As before, the polymerisable composition of the present invention may be polymerised in the mould to form the polymer. As before, an uncured polymer may be placed in the mould and cured, as an alternative method.

The mould design may encompass the anterior and/or posterior portion of the lens, or the complete lens. If only one lens surface is directly moulded, then the optics of the complementary surface may be subsequently formed by lathing and machine milling, either at room temperature or at a reduced temperature, as described above.

The mould design may encompass a single piece IOL design that incorporates moulded haptics or, alternatively, the haptics may be machined subsequent to the polymerisation and curing/or curing steps. Alternatively, a mould design may be used that is capable of providing a finished or semi-finished lens which is suitable for permanent attachment to haptic elements thereby to form a two or three-piece IOL design. Flushing and/or drying steps, as described above, may be included in the moulding of a partial or complete lens.

Where a partially finished lens is prepared from a moulding process, further machining steps are required to produce a complete lens. The precise machining steps to be carried out depend on what facets of the optic or haptics remain to be completed. For example, for a semi-finished lens shape with a completed first surface, a lathe-machining protocol such as the one described in steps (iv) to (xi) above may be followed. The steps generally described above for the lathe machining method can be used to machine a second complete surface and/or to mill or attach haptics to an ophthalmic lens that is moulded as a partially finished lens shape.

In another aspect of the inventions there is provided a method of forming a polymeric article by curing a linear polymer prepared from the polymerisable composition of the invention. The linear polymer is thus composed of polymeric units derived from the first monomer of the invention and optionally one or more polymeric units derived from the second, third and fourth monomers for use in the invention. The curing process may also be referred to as a crosslinking procedure.

In one embodiment, a polymer may be physically “cured” by the formation of an interpenetrating polymer network (IPN). Here the polymer is solubilised with a polymerisable monomer(s) which is/are polymerised to form a second polymer that is co-contingent with the first thereby to provide an interweaving polymer network which is essentially non-divisible (“intermingled”) due to chain entanglement. The polymers within the IPN formulation may optionally each incorporate cross-linking components so as to allow for the introduction of chemical cross-links.

In a further embodiment, a linear polymer may be formed comprising polymeric units derived from the first monomer of the invention. The linear polymer further comprises functionality that can be interlinked (“cured”) in a subsequent step. The functionality may be present in the polymeric units derived from the first monomer of the invention, and/or it may be present in one or more of the polymeric units derived from the second, third or fourth monomers, where present.

One example of a reactive monomer suitable for incorporation into a polymer that is to be cured is glycidyl methacrylate. When contained within a polymer, the epoxide functionality of this monomer is capable of forming interlinks with adjacent polymer chains by reaction with a suitable dinucleophile. Examples include, but are not limited to, an alkyldialkoxide, an alkyldimercaptan, an alkyldiamine, an alkyldicarboxylic acid, and an alkyldicarboxylate salt.

An alternative approach is to incorporate into a linear polymer functionality that is capable of photo-crosslinking One example of a photoreactive monomer suitable for incorporation into a polymer that is to be cured is 9-anthracene methyl methacrylate. When contained within a polymer, the photoreactive moiety undergoes light-induced 4π+4π cycloaddition with an adjacent anthracene ring to form a dianthracene linkage.

Where a polymer contains aryl groups, the aryl groups may be crosslinked by reaction with a dihalogen compound under electrophilic aromatic substitution conditions, for example using the Friedel-Craft alkylation/acylation reaction.

Aryl groups in a polymer may be reacted under Blanc conditions to generate the appropriate arylmethylene chlorides. The chloromethyl groups may be reacted with a dinucleophile to form the crosslinks. Suitable dinucleophiles include, but are not limited to, alkyldialkoxide, alkyldimercaptan, alkyldiamine, alkyldicarboxylic acid, and alkyldicarboxylate salt. Alternatively the chloromethyl groups may be reacted with the potassium salt of maleimide and the resultant arylmethylenemaleimide may be permitted to undergo cross-linking via a photo-crosslinking mechanism and/or free-radical vinyl-type polymerisation, as appropriate.

### Absorption

Typically each of —Ar¹ and —Ar² is a group which absorbs a negligible amount of light having a wavelength in the range 300-900 nm. Typically the groups are selected such that a polymer, blank or lens comprising these groups absorbs a negligible amount of light at wavelengths in that range.

Any other aryl groups present in the monomer molecule, or present in the polymerisable composition, may also absorb light at a negligible amount at a wavelength in the range 300-900 nm. However, where the polymerisable composition comprises UV-blocker components, such components are provided specifically to absorb and dissipate radiative energy from the UVA and UVB spectral ranges.

Preferably the monomer absorbs a negligible amount of light having a wavelength in the range 300-900 nm. Thus, —Ar¹ and —Ar² together with other functionality in the monomer molecule absorb a negligible amount of light having a wavelength in the range 300-900 nm.

Preferably the polymer prepared from a polymerisable composition comprising the monomer absorbs a negligible amount of light having a wavelength in the range 300-900 nm (with the aforementioned exception of any UV-blocker monomer component). Thus, the monomer (also referred to as the first monomer) and the second, third and fourth monomers, where present, and the conventional compounds, where present, and the diluent, where present, absorb a negligible amount of light having a wavelength in the range 300-900 nm.

The absorbance value may be expressed as a transmittance value.

In one embodiment, —Ar¹ and —Ar², or the monomer, or the polymer, do not significantly absorb light at a wavelength in the range 300 to 900 nm.

In one embodiment, the range is 300 to 400 nm.

In one embodiment, the range is 320 to 400 nm.

In one embodiment, the wavelength is selected from one or more of 310, 320, 330, 340, 350, 400, 450, 500, 550, 600, 700, 800 or 900 nm.

In one embodiment, —Ar¹ and —Ar², or the monomer, or the polymer, has a transmittance of at least 60%, at least 70%, at least, 80%, or at least 90% at the wavelength specified.

The phrase significantly absorb light may be taken to refer to the wavelength at which the group, monomer or polymer in question has its maximum UV absorbance (or minimum UV transmittance). In some embodiments, therefore, where the maximum UV absorbance lies outside the range 300 to 900 nm (for example, 200 to 300 nm), that group, monomer or polymer may be considered not to significantly absorb light at a wavelength in the range 300 to 900 nm.

FIG. 1 shows the transmittance profile of samples of ethylbenzene and thioanisole, and also the transmission curve of the human cornea.

### EMBODIMENTS

Various embodiment of the invention are set out below. Each and every compatible combination of the embodiments described is explicitly disclosed herein, as if each and every combination was individually and explicitly recited.

The embodiments described below apply to the monomer compound of formula (I) and the polymer compound comprising units of formula (II), where appropriate.

In one embodiment, —R¹ is independently —H or C₁₋₆ alkyl. In one embodiment, —R¹ is independently —H or -Me.

Where —R¹ is independently —H, the monomer or polymer may be referred to as an acrylate-based monomer or polymer.

Where —R¹ is independently -Me, the monomer or polymer may be referred to as an methacrylate-based monomer or polymer.

Preferably, —R¹ is independently —H.

In one embodiment, —Z— is independently —O—.

In one embodiment, —Z— is independently —NH— or —NR—.

In one embodiment, —R is independently optionally substituted alkyl.

In one embodiment, —R is independently alkyl.

In one embodiment, —R is independently -Me or -Et.

In one embodiment, —R² is independently —H or optionally substituted alkyl.

In one embodiment, —R² is independently —H.

In one embodiment, —R² is independently alkyl optionally substituted with aryl (arylalkyl). The aryl group may itself be optionally substituted.

In one embodiment, x and y are the same.

In one embodiment, x and y are each 1, 2 or 3.

In one embodiment, x and y are each 1 or 2.

In one embodiment, x and y are each 1.

In another embodiment, the optional substituents for Ar¹ and —Ar² are one or more groups selected from halo, alkyl, aryl, heterocyclyl, arylalkyl, heterocycyl-alkyl, alkoxy, aryloxy, aryl ether, and alkylaryl.

Each of the substituents may themselves be optionally substituted, where appropriate.

In another embodiment, the optional substituents for Ar¹ and —Ar² are one or more groups selected from halo, alkyl, heterocyclyl, arylalkyl, heterocycyl-alkyl, and alkoxy.

In one embodiment, each of A¹ and —Ar² is independently optionally substituted C₅₋₆ aryl.

In one embodiment, one or each of A¹ and —Ar² are unsubstituted C₅₋₆ aryl.

In one embodiment, each C₅₋₁₀ aryl is a C₅₋₆ aryl.

In one embodiment, each C₅₋₁₀ aryl is a C₆₋₁₀ carboaryl.

In one embodiment, —Ar¹ and —Ar² are each independently optionally substituted phenyl.

In one embodiment, —Ar¹ and —Ar² are each independently phenyl substituted at the 4-position.

In one embodiment, —Ar¹ and —Ar² are each independently phenyl.

In an alternative aspect of the invention, x and y are each independently 0 to 4, with the proviso that x and y are not both 0. According to this aspect, one of x and y may be 0, and the other may be 1, 2 or 3, preferably 1 or 2, and most preferably 1.

### DEFINITIONS

Substituents are defined and exemplified below.

The phrase “optionally substituted” as used herein, pertains to a parent group which may be unsubstituted or which may be substituted.

Unless otherwise specified, the term “substituted” as used herein, pertains to a parent group which bears one or more substituents. The term “substituent” is used herein in the conventional sense and refers to a chemical moiety which is covalently attached to, or if appropriate, fused to, a parent group. A wide variety of substituents are well known, and methods for their formation and introduction into a variety of parent groups are also well known. The substituents may be selected from the groups listed below.

Alkyl: The term “alkyl” as used herein, pertains to a monovalent moiety obtained by removing a hydrogen atom from a carbon atom of a saturated hydrocarbon compound, which may be aliphatic or alicyclic (cycloalkyl). The alkyl group may be a C₁₋₂₀, C₁₋₁₀, C₃₋₂₀, C₈₋₂₀, C₁₀₋₂₀, C₃₋₁₀, C₁₋₈, C₃₋₈, C₁₋₆, C₃₋₆, C₁₋₅, C₃₋₅, C₁₋₄, or C₁₋₂ alkyl group. A preferred aliphatic alkyl group is C₁₋₁₀ alkyl, most preferably C₁₋₆ alkyl. A preferred cycloalkyl group is C₃₋₁₀ cycloalkyl, most preferably C₃₋₆ cycloalkyl.

Examples of alkyl groups include, but are not limited to, methyl (C₁), ethyl (C₂), propyl (C₃), butyl (C₄), pentyl (C₅), hexyl (C₆), heptyl (C₇) and octyl (C₈).

An example of a substituted alkyl group includes, but is not limited to, perfluorooctyl (C₈F₁₇).

Examples of linear alkyl groups include, but are not limited to, methyl (C₁), ethyl (C₂), n-propyl (C₃), n-butyl (C₄), n-pentyl (amyl) (C₅), n-hexyl (C₆), n-heptyl (C₇) and n-octyl (C₈).

Examples of branched alkyl groups include iso-propyl (C₃), iso-butyl (C₄), sec-butyl (C₄), tert-butyl (C₄), iso-pentyl (C₅), and neo-pentyl (C₅).

Examples of cycloalkyl groups include, but are not limited to, those derived from:
saturated monocyclic hydrocarbon compounds:
   cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇) and methylcyclohexane (C₇); and
saturated polycyclic hydrocarbon compounds:
   norcarane (C₇), norpinane (C₇), norbornane (C₇).

Alkenyl: The term “alkenyl” as used herein, pertains to a monovalent moiety obtained by removing a hydrogen atom from a carbon atom of an unsaturated hydrocarbon compound having one or more carbon-carbon double bonds, which may be aliphatic or alicyclic (cycloalkenyl). The alkenyl group may be a C₂₋₂₀, C₂₋₁₀, C₃₋₂₀, C₃₋₁₀, C₂₋₆ or C₃₋₆ alkenyl group.

Examples of alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

An example of a substituted alkenyl group includes, but is not limited to, styrene (—CH═CHPh or —C(Ph)═CH₂).

Examples of cycloalkenyl groups include, but are not limited to, those derived from cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇) and methylcyclohexene (C₇).

Alkynyl: The term “alkynyl” as used herein, pertains to a monovalent moiety obtained by removing a hydrogen atom from a carbon atom of an unsaturated hydrocarbon compound having one or more carbon-carbon triple bonds, which may be aliphatic or alicyclic (cycloalkynyl). The alkynyl group may be a C₂₋₂₀, C₂₋₁₀, C₃₋₂₀, C₃₋₁₀, C₂₋₆ or C₃₋₆ alkenyl group.

Examples of alkynyl groups include, but are not limited to, ethynyl (ethinyl, —C≡CH) and 2-propynyl (propargyl, —CH₂—C≡CH).

Heterocyclyl: The term “heterocyclyl” as used herein, pertains to a monovalent moiety obtained by removing a hydrogen atom from a ring atom of a heterocyclic compound. The heterocyclyl group may be a C₃₋₂₀ heterocyclyl group of which from 1 to 10 are ring heteroatoms, a C₃₋₇ heterocyclyl group of which from 1 to 4 are ring heteroatoms, or a C₅₋₆ heterocyclyl group of which 1 or 2 are ring heteroatoms. In one embodiment, the heterocyclyl group is a C₃ heterocyclyl group. In one embodiment, the heterocyclyl group is epoxy. In one embodiment, the heterocyclyl group is obtained by removing a hydrogen atom from a ring carbon atom of a heterocyclic compound.

In one embodiment, the heteroatoms may be selected from O, N or S. In one embodiment the heterocyclyl group is obtained by removing a hydrogen atom from a ring nitrogen atom, where present, of a heterocyclic compound. The heterocyclyl group may be a C₃₋₂₀, C₃₋₇, or C₅₋₆ heterocyclyl group.

In this context, the prefixes (e.g. C₃₋₂₀, C₃₋₇, C₅₋₆, etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);
O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);
S₁: thiirane (C₃), thietane (C₄), thiolane (tetrahydrothiophene) (C₅), thiane (tetrahydrothiopyran) (C₆), thiepane (C₇);
O₂: dioxolane (C₅), dioxane (C₆), and dioxepane (C₇);
O₃: trioxane (C₆);
N₂: imidazolidine (C₅), pyrazolidine (diazolidine) (C₅), imidazoline (C₅), pyrazoline (dihydropyrazole) (C₅), piperazine (C₆);
N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);
N₁S₁: thiazoline (C₅), thiazolidine (C₅), thiomorpholine (C₆);
N₂O₁: oxadiazine (C₆);
O₁S₁: oxathiole (C₅) and oxathiane (thioxane) (C₆); and,
N₁O₁S₁: oxathiazine (C₆).

Examples of substituted monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

Aryl: The term “aryl”, as used herein, pertains to a monovalent moiety obtained by removing a hydrogen atom from an aromatic ring atom of an aromatic compound. It is preferred that aryl groups present in the monomers, compositions and polymers of the invention absorb a negligible amount of light having a wavelength in the range 300-900 nm. The aryl group may be a C₅₋₆ aryl group. Alternatively, the aryl group may be a C₅₋₉ aryl group or a C₅₋₁₀ aryl group.

In this context, the prefixes denote the number of ring atoms, or range of number of ring atoms, whether carbon atoms or heteroatoms. For example, the term “C₅₋₆ aryl” as used herein, pertains to an aryl group having 5 or 6 ring atoms.

The ring atoms may be all carbon atoms, as in “carboaryl groups”.

Examples of carboaryl groups include, but are not limited to, those derived from benzene (i.e. phenyl) (C₆), naphthalene (C₁₀), and azulene (C₁₀). Examples of aryl groups which comprise fused rings, at least one of which is an aromatic ring, include, but are not limited to, groups derived from indane (e.g. 2,3-dihydro-1H-indene) (C₉), indene (C₉), isoindene (C₉), and tetraline (1,2,3,4-tetrahydronaphthalene (C₁₀).

Alternatively, the ring atoms may include one or more heteroatoms, as in “heteroaryl groups”. Examples of monocyclic C₅₋₆ heteroaryl groups include, but are not limited to, those derived from:

N₁: pyrrole (azole) (C₅), pyridine (azine) (C₆);
O₁: furan (oxole) (C₅);
S₁: thiophene (thiole) (C₅);
N₁O₁: oxazole (C₅), isoxazole (C₅), isoxazine (C₆);
N₂O₁: oxadiazole (furazan) (C₅);
N₃O₁: oxatriazole (C₅);
N₁S₁: thiazole (C₅), isothiazole (C₅);
N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);
N₃: triazole (C₅), triazine (C₆); and,
N₄: tetrazole (C₅).

Examples of heteroaryl which comprise fused rings, include, but are not limited to:

C₉ (with 2 fused rings) derived from benzofuran (O₁), isobenzofuran (O₁), indole (N₁), isoindole (N₁), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂5); C₁₀ (with 2 fused rings) derived from chromene (OA isochromene (OA chroman (O₁), isochroman (O₁), benzodioxan (O₂), quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄).

Arylalkyl: The term “arylalkyl” or “aralkyl”, as used herein, pertains to a monovalent moiety obtained by removing a hydrogen atom from a carbon atom of an alkyl group that is covalently bonded to an aromatic ring. The alkyl and aryl part of the group are as defined above. The arylalkyl group may be C₆₋₂₁, C₆₋₁₃, C₆₋₈, or C₆₋₇ arylalkyl group.

The prefixes (e.g. C₆₋₂₁, C₆₋₁₃, C₆₋₈, etc.) denote the number of carbon atoms in the alkyl group and the total number of ring atoms. For example, the term “C₈ arylalkyl” as used herein, pertains to an arylalkyl group where the aryl group has 5 or 6 ring atoms and the alkyl chain has 2 or 3 carbon atoms. Typically the alkyl group has 1 or 2 carbon atoms. An example of an arylalkyl group includes, but is not limited to, benzyl (—CH₂Ph).

Alkylaryl: The term “alkylaryl” as used herein, pertains to a monovalent moiety obtained by removing a hydrogen atom from a carbon atom of an aryl group that is covalently bonded to an alkyl group. The alkyl and aryl part of the group are as defined above. The alkylaryl group may be C₆₋₂₁, C₆₋₁₃, C₆₋₈, or C₆₋₇ arylalkyl group.

The prefixes (e.g. C₆₋₂₁, C₆₋₁₃, C₆₋₈, etc.) denote the number of carbon atoms in the alkyl group and the total number of ring atoms. For example, the term “C₈ alkylaryl” as used herein, pertains to an alkylaryl group where the aryl group has 5 or 6 ring atoms and the alkyl chain has 2 or 3 carbon atoms. Typically the alkyl group has 1 or 2 carbon atoms. An example of an arylalkyl group includes, but is not limited tolyl (-PhMe).

Aryl ether: The term “aryl ether” as used herein, pertains to a monovalent moiety obtained by removing a hydrogen atom from the carbon atom of the alkyl group in an aryl-alkyl-ether compound having from 6 to 37 atoms, i.e. compounds of the form R—O—Z, where R is an alkyl group and Z is an aryl group, both alkyl and aryl groups are as defined above. The alkyl group in the parent aryl-alkyl-ether compound may be linear or branched. The aryl group in the aryl-alkyl-ether is as defined above.

The term “aryl ether” as used herein, also pertains to a monovalent moiety obtained by removing a hydrogen atom from the carbon atom of the alkyl group in an aryl-alkyl-ether compound having from 6 to 37 carbon atoms where the aryl group is attached to the oxygen atom of the alkyl-ether (alkoxy) unit by a methylene group, i.e. compounds of the form R—O—CH₂—Z, where R is an alkyl group and Z is an aryl group, both alkyl and aryl groups are as defined above. The methylene group connecting the aryl group (Z) to the oxygen atom of the ether unit may be substituted by an alkyl group, as defined above. The aryl group is as defined above.

Examples of aryl ether groups include, but are not limited to, the following carboaryl ethers: phenoxymethyl (PhOCH2-) (C7), 2-phenoxyethyl (PhOCH2CH2-) (C8), 1-phenoxyethyl (PhOCH(CH3)-) (C8), benzyloxymethyl (PhCH2OCH2-) (C8), (1-phenyl-1-ethyl)oxymethyl (PhCH(CH3)OCH2-) (C9) etc.

Heterocyclyl-alkyl: The term “heterocyclyl-alkyl”, as used herein, pertains to a monovalent moiety obtained by removing a hydrogen atom from a carbon atom of alkyl group that is covalently bonded to a heterocyclic compound. The heterocyclic ring or heterocyclyl group is as defined above and may have from 3 to 20 ring atoms, of which from 1 to 10 are ring heteroatoms. Preferably, each ring has from 3 to 7 ring atoms, of which from 1 to 4 are ring heteroatoms.

In this context, the prefixes (e.g. C₆₋₇ etc.) denote the number of carbon atoms in the alkyl group and the total number of ring atoms, whether carbon atoms or heteroatoms. For example, the term “C₆₋₇ heterocyclyl”, as used herein, pertains to a heterocyclyl group having 5 or 6 ring atoms and an alkyl group having 1 or 2 carbon atoms.

The above groups, whether alone or part of another substituent, may themselves optionally be substituted with one or more groups selected from themselves and the substituents listed below. Where a reference is made to optional substituents, those substituents may be selected from the groups listed above and below, or the groups above, or the groups below.

Acyl (keto): —C(═O)R, wherein R is an acyl substituent, for example, an alkyl group (also referred to as alkylacyl or alkanoyl), a heterocyclyl group (also referred to as heterocyclylacyl), or an aryl group (also referred to as arylacyl), preferably an alkyl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).

Acylamido (acylamino): —NR¹C(═O)R², wherein R¹ is an amide substituent, for example, hydrogen, an alkyl group, a heterocyclyl group, or an aryl group, preferably hydrogen or an alkyl group, and R² is an acyl substituent, for example, an alkyl group, a heterocyclyl group, or an aryl group, preferably hydrogen or an alkyl group. Examples of acylamide groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl, and phthalimidyl:

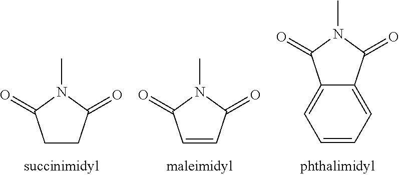

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

Amino: —NR¹R², wherein R¹ and R² are independently amino substituents, for example, hydrogen, an alkyl group (also referred to as alkylamino or dialkylamino), an alkenyl group, an alkynyl group, a heterocyclyl group, or an aryl group, preferably H or an alkyl group, or, in the case of a “cyclic” amino group, R¹ and R², taken together with the nitrogen atom to which they are attached, form a heterocyclic ring having from 4 to 8 ring atoms. Amino groups may be primary (—NH₂), secondary (—NHR¹), or tertiary (—NHR¹R²), and in cationic form, may be quaternary (—⁺NR¹R²R³). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Aminocarbonyloxy: —OC(═O)NR¹R², wherein R¹ and R² are independently amino substituents, as defined for amino groups. Examples of aminocarbonyloxy groups include, but are not limited to, —OC(═O)NH₂, —OC(═O)NHMe, —OC(═O)NMe₂, and —OC(═O)NEt₂.

Anhydride: —C(═O)OC(═O)R, wherein R is independently an anhydride substituent, for example an alkyl group, an alkenyl group, an alkynyl group, a heterocyclyl group, or an aryl group, preferably an alkyl group.

Cyanato: —OCN.

Cyano (nitrile, carbonitrile): —CN.

Ester: —C(═O)OR (carboxylate, carboxylic acid ester, oxycarbonyl) or —OC(═O)R (acyloxy, reverse eter), wherein R is an ester substituent, for example, an alkyl group, an alkenyl group, an alkynyl group, a heterocyclyl group, or an aryl group, preferably an alkyl group or an alkenyl group, most preferably an alkenyl group.

Examples of ester groups include, but are not limited to, —C(═O)OCH₃, —C(═O)OCH₂CH₃, —C(═O)OC(CH₃)₃, and —C(═O)OPh.

Other examples of ester groups include, but are not limited to, —OC(═O)CH₃ (acetoxy), —OC(═O)CH₂CH₃, —OC(═O)C(CH₃)₃, —OC(═O)Ph, —OC(═O)CH₂Ph, —OC(═O)CH═CH₂ (acrylate) and —OC(═O)C(CH₃)═CH₂ (methacrylate).

Ether: —OR, wherein R is an ether substituent, for example, an alkyl group (referred to as alkoxy), an arylalkyl group, an alkenyl group, an alkynyl group, a heterocyclyl group, or an aryl group (referred to as aryloxy), preferably an alkyl group, an arylalkyl group, or an aryl group. Examples of ether groups include, but are not limited to, —OCH₃, —OCH₂CH₃, —O-t-Bu, —OBn, and —OPh.

Formyl (carbaldehyde, carboxaldehyde): —C(═O)H.

Halo: —F, —Cl, —Br, and —I.

Hydroxy: —OH.

Isocyanato: —NCO.

Mercapto: —SR, wherein R is a mercapto substituent, for example, —H, an alkyl group, an alkenyl group, an alkynyl group, a heterocyclyl group, or an aryl group, preferably —H, an alkyl group, or an aryl group. Examples of mercapto groups include, but are not limited to, —SH, —SCH₃, —SCH₂CH₃, —S-t-Bu, and —SPh.

Nitro: —NO₂.

Phosphino (phosphine): —PR₂, wherein R is a phosphino substituent, for example, —H, an alkyl group, an alkenyl group, an alkynyl group, a heterocyclyl group, or an aryl group, preferably —H, an alkyl group, or an aryl group. Examples of phosphino groups include, but are not limited to, —PH₂, —P(CH₃)₂, —P(CH₂CH₃)₂, —P(t-Bu)₂, and —P(Ph)₂.

Ureido: —N(R¹)CONR²R³ wherein R² and R³ are independently amino substituents, as defined for amino groups, and R¹ is a ureido substituent, for example, hydrogen, an alkyl group, a heterocyclyl group, or an aryl group, preferably hydrogen or an alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, and —NMeCONEt₂.

The invention will now be further described with reference to the following non-limiting Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### EXPERIMENTAL

### Monomer Preparation

### Preparation of 1,5-Diphenylpentan-3-yl Acrylate (DPPA)

### Synthesis of 1,5-diphenylpentan-3-ol (1)

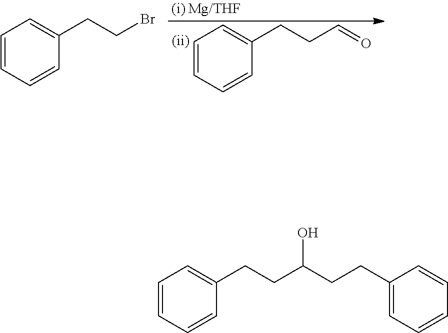 [SMILES: BrCCc1ccccc1 ; O=CCCc1ccccc1 ; OC(CCc1ccccc1)CCc1ccccc1]

Magnesium turnings (99.8% pure, 5.00 g, 0.206 moles) were placed into a dry (heat-gun dried under vacuum) 3-neck 500 mL round-bottomed flask attached to a double-layer coil condenser (side-arm), a 125 mL pressure-equalising funnel (centre-socket), a suba-seal (side-arm) and a vacuum-nitrogen manifold and purge-filled with nitrogen. 2-(Bromoethyl)benzene (25.0 mL, 0.183 moles) was measured into a dry 250 mL 3-neck round-bottomed flask. This flask was purge-filled with nitrogen and 100 mL of anhydrous tetrahydrofuran was cannula transferred into the flask forming a colourless solution. The (2-bromoethyl)benzene solution was then cannula transferred into the pressure-equalising addition funnel. The (2-bromoethyl)benzene in tetrahydrofuran solution was then added to the magnesium turnings at such a rate as to maintain a gentle reflux over a period of 60 minutes. The resultant grey, slightly turbid reaction mixture was then heated to reflux with a heat-gun for a period of 15 minutes before being stirred at room temperature for 60 minutes by which time most of the magnesium turnings had been consumed bar a few small shavings.

A solution of 23.0 mL (0.174 moles) of 3-phenylpropionaldehyde in 100 mL of anhydrous tetrahydrofuran was cannula transferred into the 125 mL addition funnel attached to the reaction flask and added dropwise over a 120 minute period to the previously prepared solution of phenethyl magnesium bromide in tetrahydrofuran. A mild exotherm was observed during the addition indicating that a reaction was taking place. The reaction mixture was then stirred at room temperature overnight under a nitrogen atmosphere. The reaction mixture was quenched with 30 mL of saturated aqueous ammonium chloride causing an initial exotherm (up to 45° C.) and then the precipitation of a voluminous “granular” white solid (magnesium hydroxides). The mixture was gravity filtered through a fluted filter paper and the collected solid was washed with 3×75 mL portions of tetrahydrofuran. The filtrate and washings were combined and the solvent was stripped off in vacuo using a rotary evaporator and the residual yellow liquid was taken up in 250 mL of diethyl ether forming a yellow solution containing a fine white suspended solid (presumably from a small amount of residual magnesium residues), this yellow suspension was then dried over anhydrous sodium sulfate for a period of 45 minutes. The drying mixture was then filtered and the collected solid washed with 3×40 mL portions of diethyl ether. The filtrate and washings were combined and stripped to dryness in vacuo on a rotary evaporator to yield a yellow liquid. This liquid was then fractionally distilled in vacuo through a 5 cm Vigreux column:

FRACTION 1: 62-137° C. (0.365 torr)—colourless liquid (pre-fraction, discarded).

FRACTION 2: 137-138° C. (0.310 torr)—colourless liquid that “froze” to a lump on standing.

The receiver flask containing FRACTION 2 was sealed up until required for the next stage of the synthesis. Yield: 26.65 g (67.4%). ¹H NMR [200 MHz, CDCl₃]: 1.41 ppm (1H, d, —OH); 1.70-1.88 (4H, m, 2,4-[>CH₂]); 2.60-2.84 (4H, m, 1,5-[>CH₂]); 3.67 (1H, m, 3-CH(O)—); 7.13-7.33 (10H, m, ArH).

### Synthesis of 1,5-diphenylpentan-3-yl acrylate (2)

1,5-Diphenyl-3-pentanol (25.0 g, 104 mmol) was weighed into a 500 mL 3-neck round-bottomed flask. This flask was attached to a suba-seal (side-arm), 125 mL pressure-equalising addition funnel (centre-socket) and a double-layer coil condenser (side-arm) which in turn was connected to a vacuum-nitrogen manifold. The apparatus was then purge-filled with nitrogen three times. Dichloromethane (160 mL, anhydrous) was then cannula transferred into the reaction flask forming a colourless solution. Hunig's base (27.2 mL, 156.2 mmol) was added to the reaction solution via a gastight syringe. Separately freshly distilled, inhibitor-free acryloyl chloride (10.2 mL, 125.5 mmol) was measured into a 125 mL graduated Schlenk tube under nitrogen and dichloromethane (60 mL, anhydrous) was added to the Schlenk tube in 3×20 mL portions via gastight syringe dissolving the acryloyl chloride to form a colourless solution. The acryloyl chloride in dichloromethane solution was then cannula transferred into the pressure-equalising addition funnel. The reaction flask was then surrounded with a dry-ice/acetone cooling bath and the reaction mixture cooled to −78° C. The acryloyl chloride in dichloromethane solution was then added dropwise to the chilled reaction mixture under nitrogen at a rate of approximately 1 drop every second over a period of 135 minutes. The reaction mixture was then allowed to warm slowly to room temperature overnight under nitrogen. Next day the reaction flask was surrounded with an ice/water cooling bath and the reaction mixture chilled to <5° C. Methanol (50 mL) was added to the pressure-equalising addition funnel and added dropwise to the reaction mixture over a period of 45 minutes in order to quench the excess acryloyl chloride. The reaction mixture was then transferred to a 1000 mL separating funnel and extracted with 300 mL HCl (aq, 1 M), 400 mL Na₂CO₃ (aq, sat.), and 400 mL saturated brine. The layers were then partitioned and separated and the lower organic layer then dried over anhydrous magnesium sulfate for a period of 1 hour. The drying mixture was then filtered and the collected solid washed with 3×40 mL portions of dichloromethane. The filtrate and washings were combined and then carefully stripped down in vacuo (rotary evaporator) at a bath temperature of 25° C. The resultant deep yellow liquid was then fractionally distilled in vacuo through a 5 cm Vigreux column in the presence of four spatula measures of 5,5′,6.6′-tetrahydroxy-3,3,3′,3′-tetramethyl-1,1′-spirobisindane at a heating oil bath temperature of 180° C.

FRACTION 1: 90.5-137.5° C. (0.205 torr)—yellow liquid (pre-fraction, discarded).

FRACTION 2: 137.5-140.5° C. (0.195 torr)—off-white coloured liquid.

The main fraction (fraction 2) was an off-white coloured liquid with a barely discernible yellow tinge. Yield: 27.28 g (82.5%). ¹H NMR [200 MHz, CDCl₃]: 1.86-2.04 (4H, m, 2,4-[>CH₂]); 2.56-2.75 (4H, m, 1,5-[>CH₂]); 5.08 (1H, m, 3-CH(O)—); 5.83 (d, 1H, >CH═CHH); 6.14 (dd, 1H, —C(═O)CH═CHH); 6.42 (d, 1H, >CH═CHH); 7.10-7.32 (10H, m, ArH).

### Polymerisable Compositions and Polymers

### Analytical Methods

Analytical methods for assessing physical properties of polymers are described below.

### Swell Factor

The swell factor of the polymer is a measure of the degree the material expands in size when hydrated in an aqueous environment. A sample of polymer of accurately determined dimensions was placed in saline until it reached a maximum dimension. The increase in size of the sample in any axis is expressed as a function of the original dimension.

### Refractive Index

The refractive index of the polymer was determined through the use of a refractometer. Examples include a hand held unit such as the Atago R500 or a conventional Abbe type instrument such as a Bellingham & Stanley 70/80 unit.

### Mechanical Properties

The mechanical properties were determined by tensile testing of the material using a Zwick Z0.5 tensiometer equipped with a KAD-Z 100N load cell. The jaws of the tensiometer were set to 10 mm separation, and the test speed to 10 mm/min.

Test strips were cut from polymer films and individually mounted between the jaws of the tensiometer. The strip being tested is held under tension, and the force applied is gradually increased until the sample breaks. The modulus of elasticity is determined from a graphical plot of stress versus strain over the elastic region of the curve. For each material a number of strips were tested and the results averaged.

### Polymer Synthesis

### Example 1

For compositions that are difficult to lathe cut at room temperature it is desirable to produce the polymer in the form of a thin film in order that its properties can be investigated. A thin polymer film was produced through polymerisation of a composition as follows: DPPA (1.9737 g), Bisphenol A diacrylate-1EO/Phenol (BPADA) (0.0100 g), 2-[3′-2′H-benzotriazol-2′-yl)-4′-hydroxyphenyl]ethyl methacrylate [BTPEM] (0.02193 g) and 2,2′-azobisisobutyronitrile [AIBN] (4.386×10⁻³ g). Two glass plates were coated with a polyethylene sheet and a 0.5 mm thick cell was created between the polyethylene sheets using a polyethylene gasket. The coated faces of the glass sheets were clipped together using spring clips with a 22 gauge syringe needle being placed between the gasket and the polyethylene sheets. The cavity was then filled with the above formulation through the needle using a gastight syringe. Once the cavity was filled the syringe needle was removed, a final clip was used to seal the mould and the assembly was placed in an oven at 60° C. for 18 hours before the oven was ramped to a temperature of 90° C. for a period of 5 hours. The moulds were allowed to cool to room temperature before the film was removed from the mould. The polymer films were annealed in vacuo using a dry-ice/isopropanol cold-trapped vacuum oven attached to a two-stage rotary vane vacuum pump and the following program:

RAMP: to 30° C.; HOLD: 30° C. for 4 hours; RAMP: 30° C. to 110° C. at 10° C. per hour; HOLD: 110° C. for 24 hours; RAMP: 110° C. to 20° C. at 15° C. per hour.

The resulting film was colourless, optically clear, soft, easily foldable and relatively tack-free. The material did not swell in saline and did not develop glistenings after prolonged storage in physiological saline at 37° C. This material had an n_{D}^{20° C.} of 1.5733, a Young's modulus of 3.84 MPa and an elongation to break of 481%.

### Example 2

The same fabrication and processing procedure was followed as example 1 but the following polymerisable composition was used DPPA (1.9737 g), divinylbenzene [DVB] (0.0500 g), BTPEM (0.02193 g) and AIBN (4.386×10⁻³ g). The resulting film was colourless, optically clear, soft and easily foldable. The material did not swell in saline and did not develop glistenings after prolonged storage in saline at 37° C. This material had an n_{D}^{20° C.} of 1.5757, a Young's modulus of 16.46 MPa and an elongation to break of 132%.

### Example 3

The same fabrication and processing procedure was followed as example 1 but the following polymerisable composition was used DPPA (1.9737 g), BPADA (0.0500 g), BTPEM (0.02193 g) and AIBN (4.386×10⁻³ g). The resulting film was colourless, optically clear, soft and easily foldable. The material did not swell in saline and did not develop glistenings after prolonged storage in saline at 37° C. This material had an n_{D}^{20° C.} of 1.5736, a Young's modulus of 6.02 MPa and an elongation to break of 199%.

### Example 4

The same fabrication and processing procedure was followed as example 1 but the following polymerisable composition was used DPPA (1.9737 g), DVB (0.0300 g), BTPEM (0.02193 g) and AIBN (4.386×10⁻³ g). The resulting film was colourless, optically clear, soft and easily foldable. The material did not swell in saline and did not develop glistenings after prolonged storage in saline at 37° C. This material had an n_{D}^{20° C.} of 1.5745, a Young's modulus of 9.85 MPa and an elongation to break of 212%.

### Example 5

The same fabrication and processing procedure was followed as example 1 but the following polymerisable composition was used DPPA (1.9737 g), BPADA (0.0300 g), BTPEM (0.02193 g) and AIBN (4.386×10⁻³ g). The resulting film was colourless, optically clear, soft and easily foldable. The material did not swell in saline and did not develop glistenings after prolonged storage in saline at 37° C. This material had an n_{D}^{20° C.} of 1.5726, a Young's modulus of 4.91 MPa and an elongation to break of 259%.

### Example 6

The same fabrication and processing procedure was followed as example 1 but the following polymerisable composition was used DPPA (1.9737 g), 1,3,5-triallyl-1,3,5-triazine-2,4,6(1H,3H,5H)-trione [TAIC] (0.0300 g), BTPEM (0.02193 g) and AIBN (4.386×10⁻³ g). The resulting film was colourless, optically clear, soft and easily foldable. The material did not swell in saline and did not develop glistenings after prolonged storage in saline at 37° C. This material had an n_{D}^{20° C.} of 1.5725, a Young's modulus of 4.28 MPa and an elongation to break of 481%.

### Example 7

The same fabrication and processing procedure was followed as example 1 but the following polymerisable composition was used DPPA (1.9737 g), TAIC (0.0100 g), BTPEM (0.02193 g) and AIBN (4.386×10⁻³ g). The resulting film was colourless, optically clear, soft and easily foldable. The material did not swell in saline and did not develop glistenings after prolonged storage in saline at 37° C. This material had an n_{D}^{20° C.} of 1.5724, a Young's modulus of 6.31 MPa and an elongation to break of 427%.

### Example 8

The same fabrication and processing procedure was followed as example 1 but the following polymerisable composition was used DPPA (1.9737 g), TAIC (0.0500 g), BTPEM (0.02193 g) and AIBN (4.386×10⁻³ g). The resulting film was colourless, optically clear, soft and easily foldable. The material did not swell in saline and did not develop glistenings after prolonged storage in saline at 37° C. This material had an n_{D}^{20° C.} of 1.5719, a Young's modulus of 6.21 MPa and an elongation to break of 387%.

### Example 9

The same fabrication and processing procedure was followed as example 1 but the following polymerisable composition was used DPPA (2.2500 g), 1,4-butanediol diacrylate [BDDA] (0.0500 g), BTPEM (0.02500 g) and AIBN (5.00×10⁻³ g). The resulting film was colourless, optically clear, soft and easily foldable. The material did not swell in saline and did not develop glistenings after prolonged storage in saline at 37° C. This material had an n_{D}^{20° C.} of 1.5702, a Young's modulus of 3.62 MPa and an elongation to break of 141%.

Table 1 summarises the details of the formulations of the polymer compositions detailed in examples 1 to 9 together with their physical characterisation parameters; n_{D}^{20° C.}, Young's modulus and elongation to break.

TABLE 1
Composition and Physical Properties of Example Polymer Compositions

| | Example Number | | | | | | | | |
|---|---|---|---|---|---|---|---|---|---|
| | 1 | 2 | 3 | 4 | 5 | 6 | 7 | 8 | 9 |
| Monomer (Wt %) | | | | | | | | | |
| DPPA | 98.408 | 96.484 | 96.484 | 97.436 | 97.436 | 97.436 | 98.408 | 96.484 | 96.774 |
| BTPEM | 1.093 | 1.072 | 1.072 | 1.083 | 1.083 | 1.083 | 1.093 | 1.072 | 1.075 |
| DVB | | 2.444 | | 1.481 | | | | | |
| BPADA | 0.499 | | 2.444 | | 1.481 | | | | |
| TAIC | | | | | | 1.481 | 0.499 | 2.444 | |
| BDDA | | | | | | | | | 2.151 |
| Initiator (wt %) | | | | | | | | | |
| AIBN | 0.219 | 0.214 | 0.214 | 0.217 | 0.217 | 0.217 | 0.219 | 0.214 | 0.215 |
| n_{D}²⁰ | 1.5733 | 1.5757 | 1.5736 | 1.5745 | 1.5726 | 1.5725 | 1.5724 | 1.5719 | 1.5702 |
| Modulus, MPa | 3.84 | 16.46 | 6.02 | 9.85 | 4.91 | 4.28 | 6.31 | 6.21 | 3.62 |
| Tensile Strength, | 3.67 | 6.30 | 4.39 | 6.00 | 4.02 | 3.67 | 3.51 | 4.08 | 2.75 |
| MPa | | | | | | | | | |
| Elongation to | 481 | 132 | 199 | 212 | 259 | 481 | 427 | 387 | 141 |
| break, % | | | | | | | | | |

We claim:

1. A monomer for a polymerisable composition, the monomer having the formula (I): 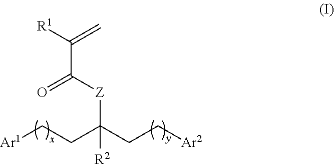
wherein:
—R¹ is —H or alkyl;
—Z— is —O—, —NH— or —NR—, where —R is optionally substituted C₁₋₆ alkyl;
—Ar¹ and —Ar² are each independently optionally substituted C₅₋₁₀ aryl;
—R² is —H, or C₁₋₄ linear or branched alkyl; and
x and y are each independently 0 to 4 with the proviso that x and y are not both 0.

2. The monomer according to claim 1, wherein each C₅₋₁₀ aryl is C₅₋₆ aryl.

3. The monomer according to claim 1 or claim 2, wherein —R² is —H.

4. The monomer according to claim 1, wherein —Z— is independently —O—.

5. The monomer according to claim 1, wherein —R¹ is independently —H or -Me.

6. The monomer according to claim 5, wherein —R¹ is independently —H.

7. The monomer according to claim 1, wherein —Ar¹ and —Ar² are each independently optionally substituted C₆₋₁₀ carboaryl.

8. The monomer according to claim 7, wherein —Ar¹ and —Ar² are each independently optionally substituted phenyl.

9. The monomer according to claim 8, wherein —Ar¹ and —Ar² are each independently phenyl.

10. A polymerisable composition comprising a first monomer having the formula (I): 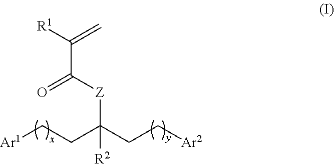
wherein:
—R¹ is —H or alkyl;
—Z— is —O—, —NH— or —NR—, where —R is optionally substituted C₁₋₆ alkyl;
—Ar¹ and —Ar² are each independently optionally substituted C₅₋₁₀ aryl;
—R² is —H, or C₁₋₄ linear or branched alkyl; and
x and y are each independently 0 to 4 with the proviso that x and y are not both 0.

11. The polymerisable composition according to claim 10, wherein the amount of first monomers in the composition is 5 to 99 wt % of the composition.

12. The polymerisable composition according to claim 10, further comprising:
one or more second monomers for polymerisation with the first monomer, wherein the second monomer has an acrylate or methacrylate group;
one or more hydrophilic third monomers for polymerisation with the first monomer; and/or
one or more fourth monomers for forming crosslinks with monomers in the polymerisable composition; and/or
a thermally- or light-activated polymerisation initiator, a UV-light absorber, or a blue-light absorber, a tackiness modifying agent, or a combination thereof, wherein the UV-light absorber and the blue-light absorber are optionally fixable.

13. A polymer obtained or obtainable from a polymerisable composition comprising a monomer having the formula (I): 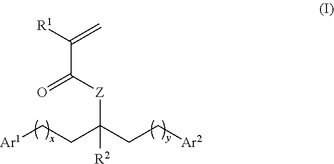
wherein:
—R¹ is —H or alkyl;
—Z— is —O—, —NH— or —NR—, where —R is optionally substituted C₁₋₆ alkyl;
—Ar¹ and —Ar² are each independently optionally substituted C₅₋₁₀ aryl;
—R² is —H, or C₁₋₄ linear or branched alkyl; and
x and y are each independently 0 to 4 with the proviso that x and y are not both 0.

14. The polymer according to claim 13 having:
a T_{g} in the range of from −50 to 35° C.; and/or
an elongation at 20° C. of at least 50%; and/or
a refractive index at 20° C. of at least 1.50.

15. A blank for an ophthalmic lens formed from the polymer according to claim 13.

16. An ophthalmic lens formed from the polymer according to claim 13.

17. The ophthalmic lens according to claim 16 which is an intraocular lens.

18. A method of forming a blank for an ophthalmic lens, the method comprising the steps of:
(a) preparing a polymer rod, wherein the polymer rod is obtained by the polymerisation of a composition in a rod-shaped mould, wherein the composition comprises a monomer having the formula (I): 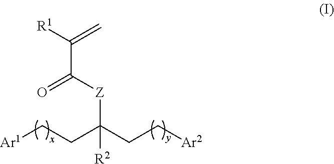
wherein:
—R¹ is —H or alkyl;
—Z— is —O—, —NH— or —NR—, where —R is optionally substituted C₁₋₆ alkyl;
—Ar¹ and —Ar² are each independently optionally substituted C₅₋₁₀ aryl;
—R² is —H, or C₁₋₄ linear or branched alkyl; and
x and y are each independently 0 to 4 with the proviso that x and y are not both 0; and
(b) working the polymer rod into a plurality of blanks; or
(a) polymerising a composition in a button mould thereby to form a lens blank, wherein the composition comprises a monomer having the formula (I): 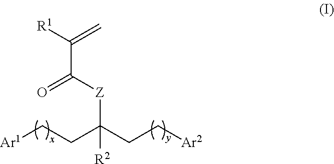
wherein:
—R¹ is —H or alkyl;
—Z— is —O—, —NH— or —NR—, where —R is optionally substituted C₁₋₆ alkyl;
—Ar¹ and —Ar² are each independently optionally substituted C₅₋₁₀ aryl;
—R² is —H, or C₁₋₄ linear or branched alkyl; and
x and y are each independently 0 to 4 with the proviso that x and y are not both 0.

19. The method of claim 18 further comprising the step of working the blank to form an ophthalmic lens.

20. A method for forming an ophthalmic lens, the method comprising the step of polymerizing a polymerizable composition according to claim 10 in a mould thereby to form an ophthalmic lens, wherein the mould is shaped so as to provide an ophthalmic lens having anterior and/or posterior portions.
